(19) United States
(12) Patent Application Publication
(10) Pub. No.: US 2010/0298783 A1
(43) Pub. Date: Nov. 25, 2010

(54) MANUAL SWITCH FOR A CLOSED SUCTION TUBE

(76) Inventor: Ti-Li Chang, Taichung Hsien (TW)

Correspondence Address: NIKOLAI & MERSEREAU, P.A., 900 SECOND AVENUE SOUTH, SUITE 820, MINNEAPOLIS, MN, 55402, US

(21) Appl. No.: 12/469,954

(22) Filed: May 21, 2009

(51) Int. Cl. A61M 5/00

(52) U.S. Cl. 604/256

(57) ABSTRACT

A manual switch has a body, a button, a valve rod and a biasing member. The body is hollow and has a connecting tube and a valve mount. The connecting tube is mounted through the body. The valve mount is mounted in the body and communicates with the connecting tube. The button is connected pivotally to the body at a pivotal position on the body. The valve rod is mounted slidably in the valve mount to close the communication of the connecting tube, is pressed by the button and has an annular recess. The annular recess is defined around the valve rod and selectively communicates with the connecting tube when the valve rod is moved. The biasing member is mounted in the valve mount and is connected to the valve rod to provide a recoil force to the valve rod.

### BACKGROUND OF THE INVENTION

1. Field of the Invention

The present invention relates to a switch, and more particularly to a manual switch for a closed suction tube for drawing secretion out from a human body.

2. Description of Related Art

A closed suction tube is always provided with a manual switch as shown in FIG. 11 to manually control the suction tube to draw secretion out from a patient. The manual switch is connected to the suction tube and a vacuum pump/compressor and has a body (60), a button (62), a valve rod (64) and a spring (66). The body (60) has a connecting tube with two ends connected respectively with the suction tube and vacuum pump/compressor. The button (62) is mounted slidably on the body (60). The valve rod (64) is movable mounted in the body (60), is released upward by the button (62) and is mounted into the connecting tube to close the communication of the connecting tube. The spring (66) is mounted in the body (60) to provide a recoil force to the valve rod (64). When the button (62) is pressed, the valve rod (64) is moved downward to communicate the suction tube and the vacuum pump/compressor via the connecting tube. Consequently, the vacuum pump/compressor can provide a vacuum drawing force to draw secretion out from the patient through the suction tube.

However, the button (62) of the conventional switch is pressed in a vertical direction, and the force for pressing the button (62) has to be large enough for completely over the tension of the biasing member (66). Consequently, the operation of the conventional switch is laborious. In addition, although some conventional manual switch may further comprises a safety device to keep the button (62) from being pressed unintentionally, but the conventional safety device has a complicated structure and is trouble in operation.

To overcome the shortcomings, the present invention tends to provide a manual switch to mitigate or obviate the aforementioned problems.

### SUMMARY OF THE INVENTION

The main objective of the invention is to provide a manual switch for a closed suction tube and being easily operated and having a labor-saving capability. The manual switch has a body, a button, a valve rod and a biasing member. The body is hollow and has two ends, a connecting tube and a valve mount. The connecting tube is mounted through the body and has two ends protruding respectively from the ends of the body. The valve mount is hollow, is formed in the body, communicates with the connecting tube and has an inner diameter. The button is connected pivotally to the body at a pivotal position on the body. The valve rod is mounted slidably in the valve mount, is pressed by the button and has a diameter and an annular recess. The diameter of the valve rod is equal to the inner diameter of the valve mount to close a communication of the ends of the connecting tube. The annular recess is defined around the valve rod and selectively communicates with the connecting tube when the valve rod is moved. The biasing member is mounted in the valve mount and is connected to the valve rod to provide a recoil force to the valve rod.

Other objects, advantages and novel features of the invention will become more apparent from the following detailed description when taken in conjunction with the accompanying drawings.

### BRIEF DESCRIPTION OF THE DRAWINGS

FIG. 1 is a perspective view of a first embodiment of a manual switch for a closed suction tube in accordance with the present invention;

FIG. 2 is an exploded perspective view of the manual switch in FIG. 1;

FIG. 3 is a cross sectional side view of the manual switch in FIG. 1 in a locked condition;

FIG. 4 is an enlarged cross sectional end view of the manual switch in FIG. 1;

FIG. 5 is an enlarged operational cross sectional end view of the manual switch in FIG. 1 when the button is pressed;

FIG. 6 is an operational cross sectional end view of the manual switch in FIG. 1 when the button is pressed after the safety device is unlocked;

FIG. 7 is a perspective view of a second embodiment of a manual switch in accordance with the present invention;

FIG. 8 is an exploded perspective view of the manual switch in FIG. 7;

FIG. 9 is a cross sectional side view of the manual switch in FIG. 7 in an operation condition;

FIG. 10 is a cross sectional side view of the manual switch in FIG. 7 in an unused condition; and

FIG. 11 is a side view in partial section of a conventional manual switch in accordance with the prior art.

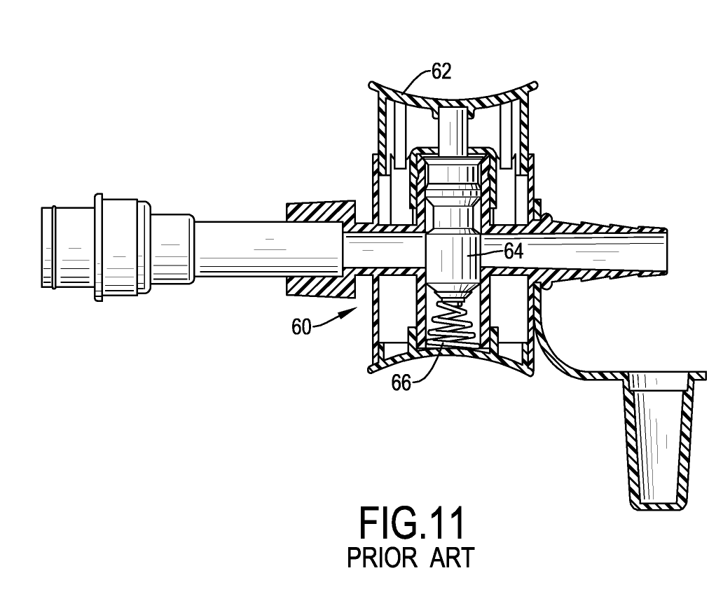

### DETAILED DESCRIPTION OF PREFERRED EMBODIMENT

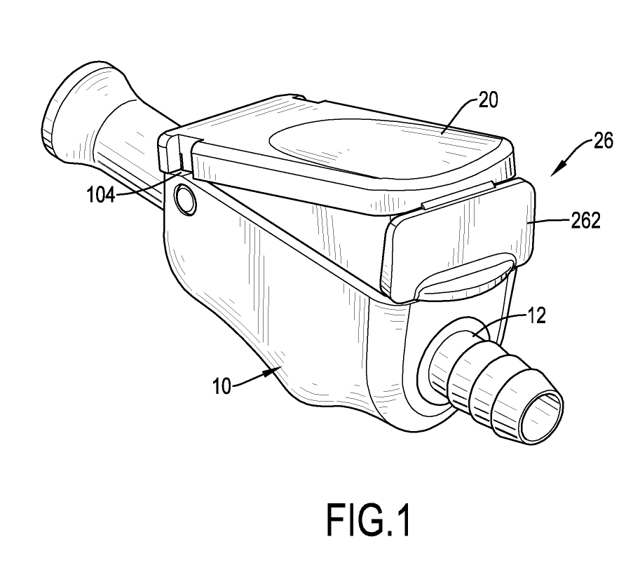

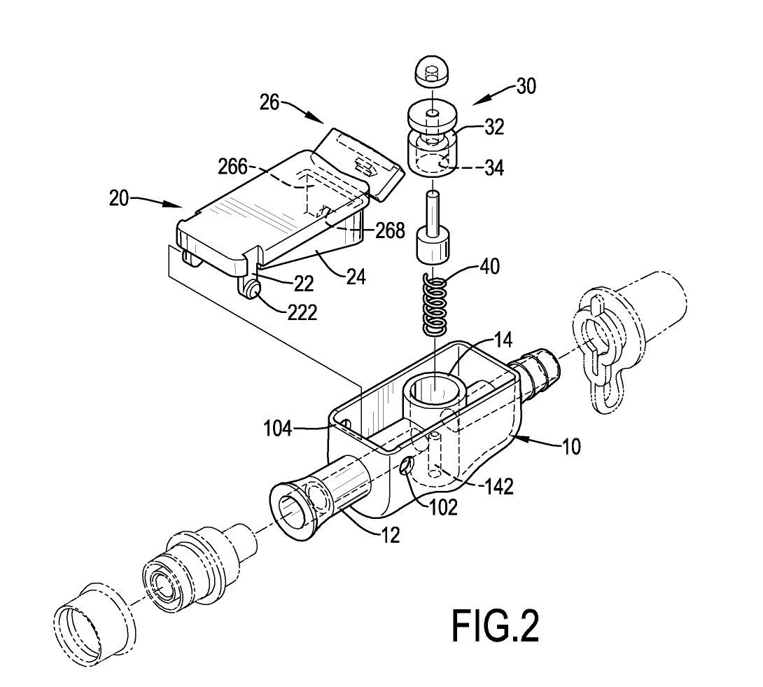

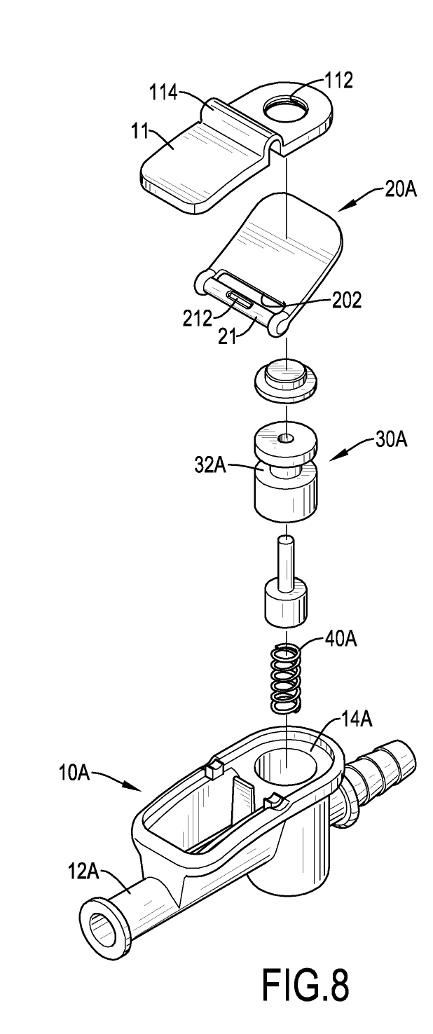

With reference to FIGS. 1, 2, 7 and 8, a manual switch for a closed suction tube in accordance with the present invention comprises a body (10,10A), a button (20,20A), a valve rod (30,30A) and a biasing member (40,40A). The body (10,10A) is hollow and has two ends, a connecting tube (12,12A) and a valve mount (14,14A). The connecting tube (12,12A) is mounted through the body (10,10A) and has two ends protruding respectively from the ends of the body (10,10A). The ends of the connecting tube (12,12A) are connected respectively to a suction tube and a vacuum pump/compressor. The valve mount (14,14A) is hollow, is formed in the body (10,10A), communicates with the connecting tube (12,12A) and has an inner diameter. The button (20,20A) is connected pivotally to the body (10,10A) at a pivotal position on the body (10,10A). The valve rod (30,30A) is mounted slidably in the valve mount (14,14A), is pressed by the button (20,20A) and comprises a diameter and an annular recess (32,32A). The diameter of the valve rod (30,30A) is equal to the inner diameter of the valve mount (14,14A) to close the communication of the ends of the connecting tube (12,12A). The annular recess (32,32A) is defined around the valve rod (30,30A) and selectively communicates with the connecting tube (12,12A) when the valve rod (30,30A) is moved. The biasing member (40,40A) may be a spring, is mounted in the valve mount (14,14A) and is connected to the valve rod (30,30A) to provide a recoil force to the valve rod (30,30A).

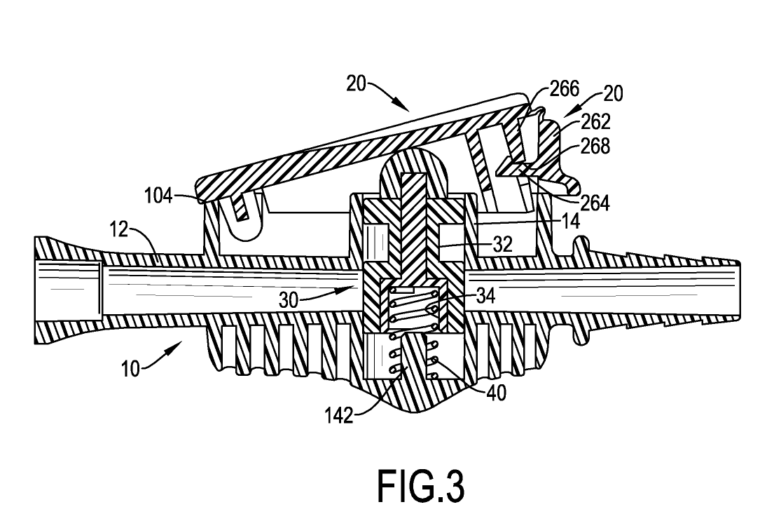

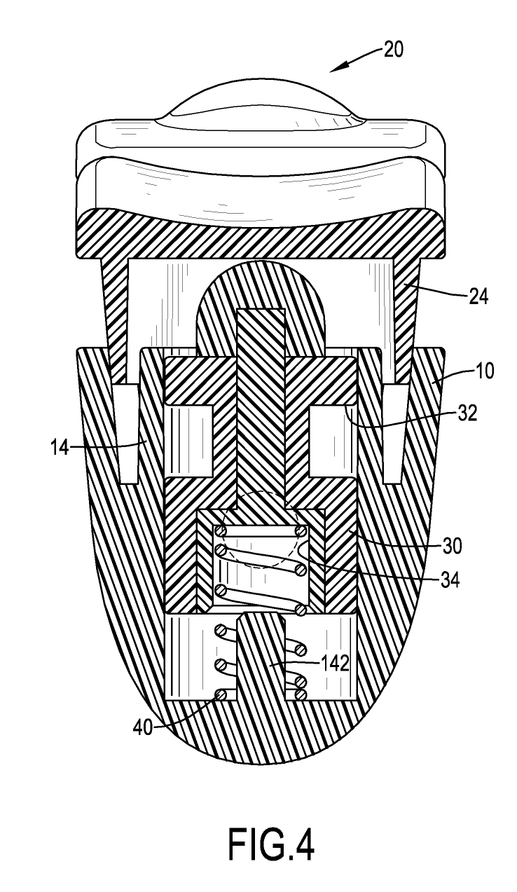

With reference to FIGS. 1 to 4, in the first embodiment, the pivotal position of the button (20) connected pivotally to the body (10) is at the top near one end of the body (10). The body (10) may further have an opening, an inner wall, two pivotal holes (102), an inclined edge (104) and a spring post (142). The opening is defined in the top of the body (10). The pivotal holes (102) are defined in or through the inner wall of the body (10). The inclined edge (104) is defined in the top of the body (10) at the end corresponding to the pivotal position. The spring post (142) is formed on and protrudes from the bottom of the valve mount (14).

The button (20) has two ends, a bottom, a wall (24) and two pivotal arms (22). One end of the button (20) is connected pivotally to the body (10) and selectively abuts with the inclined edge (104) on the top of the body (10) to limit the pivotal angle of the button (20). The wall (24) is formed on and protrudes from the bottom of the button (20) and selectively abuts with the inner wall of the body (10) to provide a guiding effect to the button (20) to make the button (20) being pressed smoothly. The pivotal arms (22) are formed on and protrude from the bottom of the button (20) at the end corresponding to the pivotal position and correspond respectively to the pivotal holes (102) in the body (10). Each pivotal arm (22) has a pivotal boss (222) formed on the pivotal arm (22) and rotatably mounted in a corresponding one of the pivotal holes (102) in the body (10) to pivotally connect the button (20) to the body (10).

The valve rod (30) may be a single piece or composed of multiple elements and has a top, a bottom, an annular recess (32) and a spring recess (34). The annular recess (32) is defined around the valve rod (30) and selectively communicates with the connecting tube (12) when the valve rod (30) is moved. The top of the valve rod (30) protrudes out from the valve mount (14) and abuts with the bottom at a position between the ends of the button (20). The spring recess (34) is defined in the bottom of the valve rod (30).

The biasing member (40) has a bottom and a top. The bottom of the biasing member (40) abuts with the bottom of the valve mount (14) and is mounted around the spring post (142). The top of the biasing member (40) abuts with the bottom of the valve rod (30) and is held in the spring recess (34) in the valve rod (30).

In addition, the manual switch may further comprise a safety device (26) mounted on the button (20) at an end opposite to the pivotal position. The safety device (26) comprises an abutting tab (262) and a locking tab (266). The abutting tab (26) is pivotally connected to the end of the button (20) opposite to the pivotal position, selectively abuts against the top of the body (10) and has a hook (264) formed on the abutting tab (262). The locking tab (266) is formed on and protrudes from the bottom of the button (20) and has a locking hole (268). The locking hole (268) is defined in the locking tab (266) and selectively engages the hook (264) on the abutting tab (262) when the abutting tab (262) is pivoted to abut against the top of the body (10). With the abutment of the abutting tab (262) against the top of the body (10), the button (20) is kept from being pivoted relative to the body (10). Accordingly, the button (20) will not be pressed unintentionally, and the safety of using the suction tube is improved.

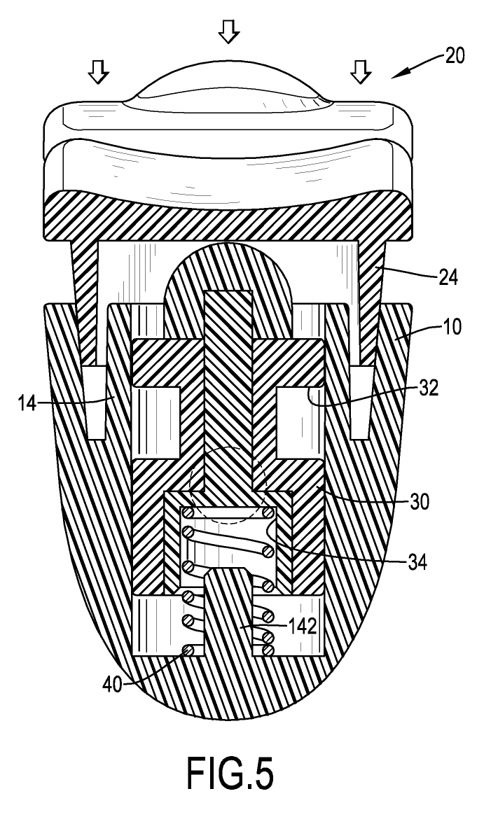

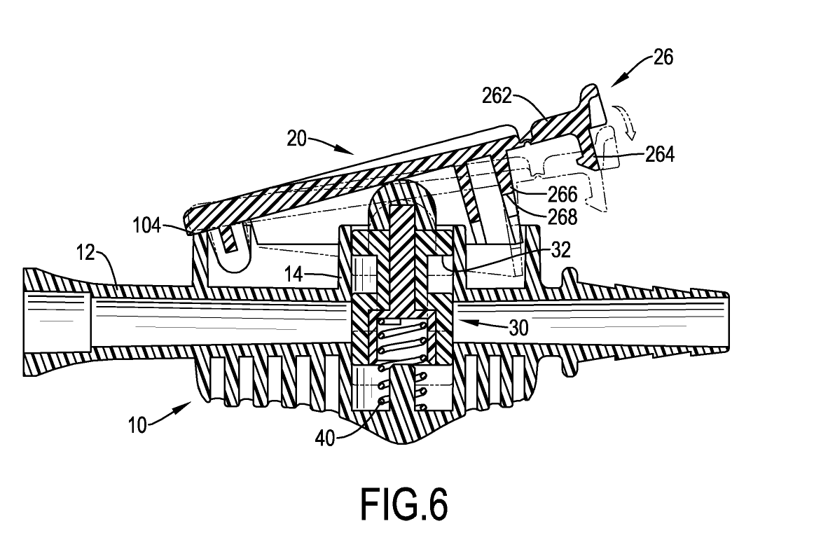

With further reference to FIGS. 5 and 6, in use, the abutting tap (262) is firstly pivoted to disengage the hook (264) from the locking hole (268) to make the button (20) being able to pivot relative to the body (10). When the button (20) is pressed, the button (20) is pivoted relative to the body (10) and the valve rod (30) is moved downward along the valve mount (14) to make the annular recess (32) communicating with the connecting tube (12). Consequently, the ends of the connecting tube (12) are communicated, such that the vacuum pump/compressor can provide a suction force to the suction tube to draw secretion out from a patient through the connecting tube (12) of the manual switch.

After the secretion-sucking process has been done, the button (20) is released, the valve rod (30) and the button (20) are moved to the original position with the recoil force provided by the biasing member (40) and the connecting tube (12) is closed by the valve rod (30) again.

Because the button (20) is connected pivotally to the body (10) and the valve rod (30) abuts with the button (20) at a location between two ends of the button (20), the pressing force applied to the button (20) will be enlarged for pressing against the valve rod (30) due to the Lever Principle. Therefore, a less force is needed for pressing the button (20) and against the tension of the biasing member (40), and the manual switch in accordance with the present invention has a labor-saving capability.

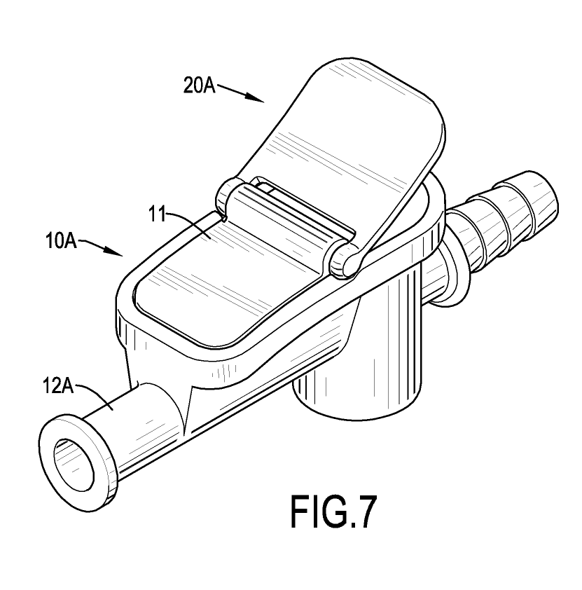

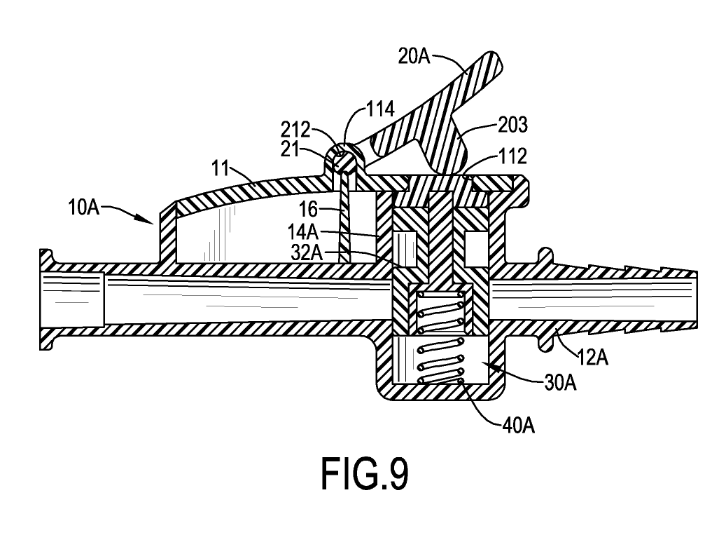

With reference to FIGS. 7 to 9, in the second embodiment, the body (10A) has a top, an opening defined in the top of the body (10A), a cover (11) attached to the top of the body (10A) to close the opening and a positioning tab (16). The cover (11) has a top surface, a middle segment, a pressing hole (112) and pivotal mount (114). The pivotal position of the button (20A) connected pivotally to the body (10A) is at the middle segment of the cover (11). The pressing hole (112) is defined through the cover (11) and aligns with the valve mount (14A) in the body (10A). The pivotal mount (114) is formed on the middle segment and is hollow. The positioning tab (16) is formed in the body (10A) and protrudes toward the cover (11).

The button (20A) is connected pivotally to the middle segment of the cover (11) with one end and has a pivotal rod (21), a slot (202) and a pressing protrusion (203). The pivotal rod (21) is formed on the end of the button (20A) corresponding to the pivotal position and is rotatably held in the pivotal mount (114) on the cover (11). The pivotal rod (21) may have multiple positioning grooves (212) defined longitudinally in the pivotal rod (21), and the positioning tab (16) in the body (10A) engages one of the positioning grooves (212) to hold the button (20A) at a desired position.

The slot (202) is defined through the button (20A) and is parallel with the pivotal rod (21), and one end of the cover (11) extends through the slot (202) for combining the button (20A) with the cover (11). The pressing protrusion (203) is formed on and protrudes from the bottom at a position between the ends of the button (20A).

The top of the valve rod (30A) protrudes into the pressing hole (112) in the cover (11), abuts with the pressing protrusion (203) on the button (20A) and may be flush with the top surface of the cover (11).

With such an arrangement, when the button (20A) is pressed and pivoted, the valve rod (30A) is moved downward along the valve mount (14A) to make the annular recess (32A) communicating with the connecting tube (12A). When the button (20A) is released, the valve rod (30A) and the button (20A) are moved to the original position with the recoil force provided by the biasing member (40A) to close the communication of the connecting tube (12A).

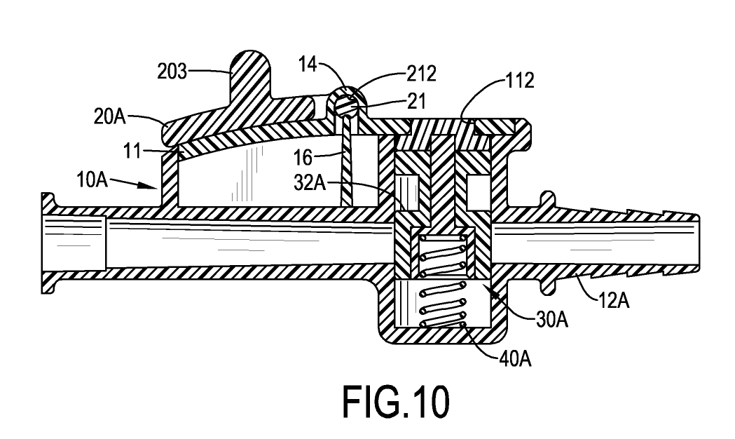

With further reference to FIG. 10, when the suction tube is not in use, the button (20A) is pivoted relative to the cover (11) to make the pressing protrusion (203) leaving away from the valve rod (30A). Accordingly, because the top of the valve rod (30A) is flush with the top surface of the cover (11), the valve rod (30A) will not be unintentionally pressed to improve the safety of using the suction tube. Therefore, the manual switch in accordance with the present invention has a safety capability with a simplified structure and a convenient operation way.

Even though numerous characteristics and advantages of the present invention have been set forth in the foregoing description, together with details of the structure and function of the invention, the disclosure is illustrative only, and changes may be made in detail, especially in matters of shape, size, and arrangement of parts within the principles of the invention to the full extent indicated by the broad general meaning of the terms in which the appended claims are expressed.

1. A manual switch for a closed suction tube comprising:
a hollow body having
two ends;
a connecting tube mounted through the body and having two ends protruding respectively from the ends of the body; and
a hollow valve mount, formed in the body, communicating with the connecting tube and having an inner diameter;
a button connected pivotally to the body at a pivotal position on the body;
a valve rod mounted slidably in the valve mount, pressed by the button and comprising
a diameter being equal to the inner diameter of the valve mount to close communication between the ends of the connecting tube; and
an annular recess defined around the valve rod and selectively communicating with the connecting tube when the valve rod is moved; and
a biasing member mounted in the valve mount and connected to the valve rod to provide a recoil force to the valve rod,
wherein the body has an inner wall, a top and an opening defined in the top of the body;
the pivotal position of the button connected pivotally to the body is at the opening and near one of the ends of the body;
wherein the button has
two ends;
a bottom; and
a wall formed on and protruding from the bottom of the button and selectively abutting with the inner wall of the hollow body;
wherein the manual switch further comprises a safety device mounted on the button at an end opposite to the pivotal position and comprising
an abutting tab pivotally connected to the end of the button opposite to the pivotal position, selectively abutting against a top of the hollow body and a hook formed on the abutting tab; and
a locking tab formed on and protruding from a bottom of the button and having a locking hole defined in the locking tab and selectively engaging the hook on the abutting tab when the abutting tab is pivoted to abut against the top of the body.

2. The manual switch as claimed in claim 1, wherein
the valve rod has a top protruding out from the valve mount and abutting with the bottom at a position between the ends of the button.

3. The manual switch as claimed in claim 2, wherein
the body further has two pivotal holes defined in the inner wall of the body; and
the button further has two pivotal arms formed on and protruding from the bottom of the button at the end corresponding to the pivotal position and corresponding respectively to the pivotal holes in the body, and each pivotal arm has a pivotal boss formed on the pivotal arm and rotatably mounted in a corresponding one of the pivotal holes in the body.

4. (canceled)

5. The manual switch as claimed in claim 3, wherein the biasing member has a bottom abutting with a bottom of the valve mount and a top abutting with a bottom of the valve rod.

6. The manual switch as claimed in claim 5, wherein
the valve mount has a spring post formed on and protruding from the bottom of the valve mount;
the valve rod has a spring recess defined in the bottom of the valve rod;
the bottom of the biasing member is mounted around the spring post; and
the top of the biasing member is held in the spring recess in the valve rod.

7. The manual switch as claimed in claim 6, wherein the body further has an inclined edge defined in the top of the body at the end corresponding to the pivotal position and selectively abutting the end of the button corresponding to the pivotal position.

8-14. (canceled)

15. The manual switch as claimed in claim 1, wherein the biasing member has a bottom abutting with a bottom of the valve mount and a top abutting with a bottom of the valve rod.

16. The manual switch as claimed in claim 15, wherein
the valve mount has a spring post formed on and protruding from the bottom of the valve mount;
the valve rod has a spring recess defined in the bottom of the valve rod;
the bottom of the biasing member is mounted around the spring post; and
the top of the biasing member is held in the spring recess in the valve rod.

17. The manual switch as claimed in claim 1, wherein the body further has an inclined edge defined in a top of the body at the end corresponding to the pivotal position and selectively abutting an end of the button corresponding to the pivotal position.
